(19) United States
(12) Patent Application Publication
(10) Pub. No.: US 2022/0003614 A1
(43) Pub. Date: Jan. 6, 2022

(54) THERMOMETER DEVICE

(71) Applicant: Jackson State University, Jackson, MS (US)

(72) Inventors: Jordan Barber, Jackson, MS (US); Chevan Baker, Kansas City, MO (US); Fred Harris, Lake, MS (US); Jann Butler, Moss Point, MS (US); Gordon Skelton, Madison, MS (US)

(73) Assignee: Jackson State University, Jackson, MS (US)

(21) Appl. No.: 17/373,374

(22) Filed: Jul. 12, 2021

Related U.S. Application Data
(63) Continuation-in-part of application No. 15/387,376, filed on Dec. 21, 2016, now abandoned.
(60) Provisional application No. 62/314,098, filed on Mar. 28, 2016.

(51) Int. Cl. G01K 7/34; G01K 1/143; A61B 5/01; A61B 5/00; A43B 3/00

(52) U.S. Cl. CPC G01K 7/34; G01K 1/143; G01K 1/045; A61B 5/6807; A43B 3/0005; A61B 5/01

(57) ABSTRACT

A device for measuring the temperature of a lower extremity of a subject in need of such monitoring. The device is especially suited for monitoring the foot temperature of a subject with diabetes or another condition that could lead to lower blood circulation and temperature in the lower extremities of the subject. Methods of preventing complications from such diseases, disorders, syndromes, or conditions are provided. The past recorded temperatures of the subject may be stored for comparisons and trends analyses.

### CROSS-REFERENCE TO RELATED APPLICATIONS

This application is a continuation-in-part of U.S. application Ser. No. 15/387,376 to Jordan Barber et al. filed on Dec. 21, 2016, which claims priority to U.S. Provisional Application No. 62/314,098 to Jordan Barber et al. filed on Mar. 28, 2016, the contents of which are incorporated herein by reference in their entirety.

### FIELD OF THE INVENTION

The present invention is generally directed toward a device for measuring the temperature of an extremity of the anatomy of a subject, and more particularly to monitoring the foot temperature of a subject with diabetes or another condition causing relatively low blood circulation and temperature in the extremities of the subject.

### BACKGROUND OF THE INVENTION

Some diseases, disorders, syndromes, and conditions cause decreased blood flow to the extremities of the body. One such disease is diabetes mellitus. A diabetic patient has abnormal (high) glucose levels in the blood, affecting its flow to the lower extremities, such as the feet and toes. The decreased blood flow can lead to serious complications, including numbness, neuropathy, pain, and even tissue damage, ulceration, amputation, and worse. The Mississippi Department of Health said the Magnolia State ranked second in the nation for prevalence of diabetes in 2012. Accordingly, four of every 1,000 deaths in Mississippi resulted from complications of the disease.

There is a need for a means of monitoring the blood flow or other related symptoms of diabetic complications in a subject's lower extremities to prevent or lessen the damage to tissue in these subjects.

### SUMMARY OF THE INVENTION

The goal of the present invention is to address the shortcomings of the prior art and to provide a device for measuring the temperature directly (and blood flow indirectly) of a lower extremity of a subject in need thereof. Accordingly, in one aspect, the present invention provides a device for measuring the temperature of at least one foot of a subject in need thereof comprising: a structural platform, a plurality of temperature sensors, a power supply, and a control box. The structural platform may be a mat, pad, or wearable pad. The plurality of temperature sensors may be thermistor sensors. The power supply may be a battery. The control box further comprises a processor and a memory storage device. The control box may further comprise a transceiver.

In another aspect, the present invention provides a method of preventing tissue damage in a subject in need thereof comprising the steps of measuring the temperature of a lower extremity of the subject in need thereof with a plurality of temperature sensors as a first data set, processing the first data set collected from the plurality of temperature sensors, measuring the temperature of the lower extremity of the subject in need thereof with the plurality of temperature sensors as a second data set, processing the second data set collected from the plurality of temperature sensors, analyzing the first and second data sets, providing an instruction for a user or caregiver to take a remedial action based on the results of the analyzing step.

### BRIEF DESCRIPTION OF THE DRAWINGS

Further advantages of the invention will become apparent by reference to the detailed description of preferred embodiments when considered in conjunction with the drawings:

FIG. 1 is a top plan view of a temperature measuring device including a mat, according to various examples.

FIG. 2A is a top plan view of a temperature measuring device including an insole, according to various examples.

FIG. 2B is a side elevation view of a temperature measuring device including a hosiery article, according to various examples.

FIG. 3 is a top plan view of an alternative sensor arrangement for a temperature measuring device.

FIG. 4 is a top perspective view of a temperature measuring device including a mat, according to various examples.

FIG. 5 is a top plan view of a portable device configured to be used with a temperature measuring device, according to various examples.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

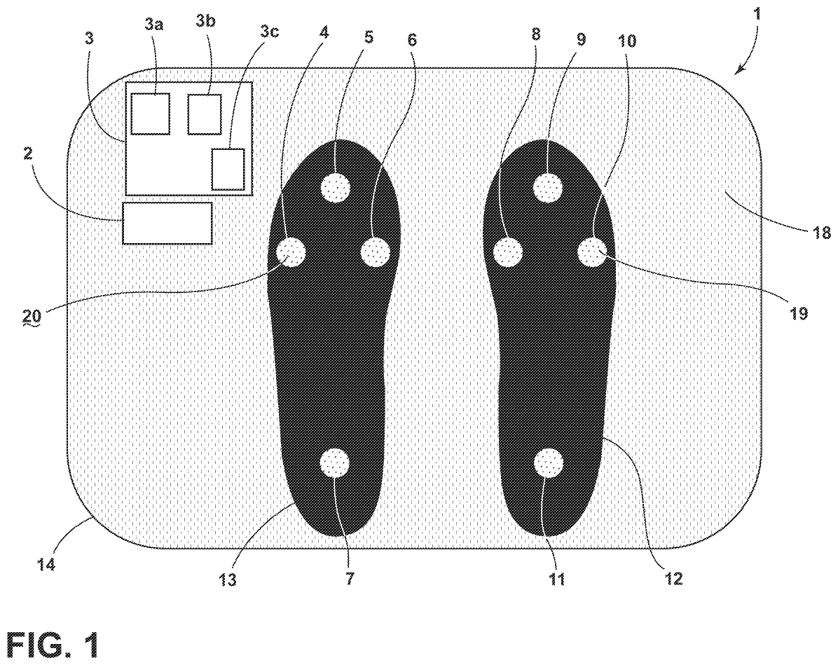

Referring now to FIGS. 1-3, reference numeral 1 generally indicates an electronic temperature measuring device. The electronic temperature measuring device 1 is configured to measure the temperature of an extremity of a subject in need thereof and to take subsequent appropriate remedial action based on the results of such measurement readings. A subject in need thereof should be understood in this context to be a person suffering from, suspected to be suffering from, and/or at risk of suffering from at least one disease, disorder, syndrome, or condition that causes at least some relative lowering of the body temperature in one or more extremities of the body, whether periodically or otherwise. Diabetes mellitus is one example of such an at least one disease, disorder, syndrome, or condition that causes at least some relative lowering of the body temperature in one or more extremities of the body. However, it is contemplated that the electronic temperature measuring device may be configured to monitor the temperature of an extremity of any individual, including those without a disease, disorder, syndrome, or condition without departing from the scope of the present disclosure.

In various examples, the electronic temperature measuring device 1 may be configured to measure the temperature of a subject's foot. As best shown in FIG. 1, the electronic temperature measuring device 1 may include a platform 14. In various examples, the platform 14 may be configured as a foot mat (e.g., an anti-fatigue foot mat), which may allow for improved ease of temperature measuring. The temperature measuring device 1 may further include a plurality of temperature sensors 4-11. In various example, one or more of the plurality of temperature sensors 4-11 may be coupled with, positioned on, or integrally formed with the platform 14. The sensors 4-11 are configured to provide for an accurate temperature reading of the subject's foot/feet temperature, as discussed in more detail elsewhere herein.

The temperature measuring device 1 may further include a right foot placement guide 12 and a left foot placement guide 13. The right and left foot placement guides may be configured to assist a user/subject to properly place the foot/feet over the plurality of temperature sensors 4-11. In other words, the plurality of temperature sensors 4-11 may be located such that one or more of the plurality of temperature sensors 4-11 are positioned in contact with a subject's foot while a subject is standing or sitting or otherwise has the subject's foot or feet in contact with the temperature measuring device 1 as illustrated by the guide 12, 13.

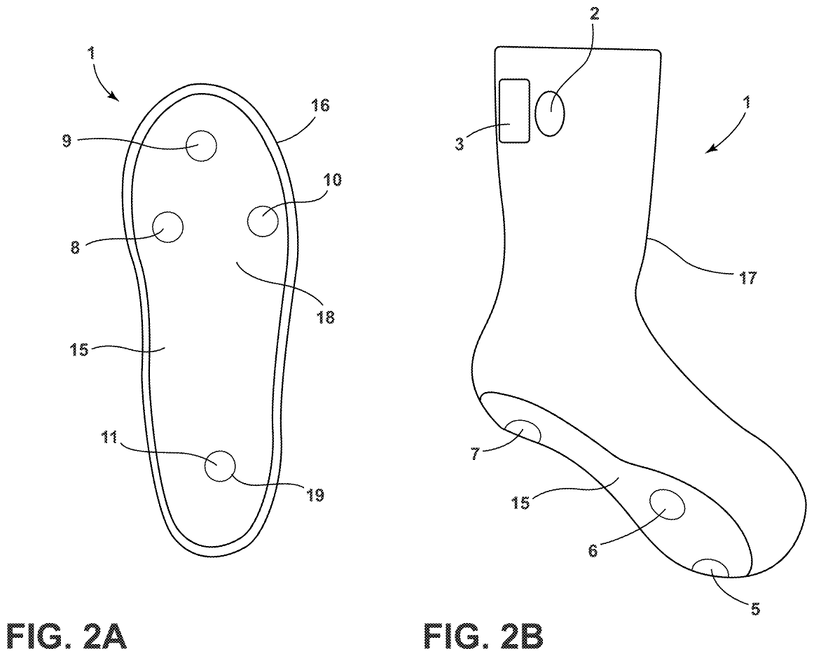

As shown in FIGS. 2A and 2B, in alternative examples, the temperature measuring device 1 may be configured as a foot pad 15. It will be understood that the temperature measuring device 1 may be configured as a single foot pad 15 or a pair of foot pads 15 without departing from the scope of the present disclosure. Where the temperature measuring device 1 is a pair of foot pads 15, it will be understood that each of the pair of foot pads 15 includes the same or similar configuration of a single foot pad 15 described herein.

Where the foot temperature measuring device 1 includes a foot pad 15 or a pair of foot pads 15, the foot temperature measuring devices 1 may be configured to be integrated into, positioned on, and/or coupled with insoles 16 (e.g., wearable insoles for shoes and other footwear) (see FIG. 2A), socks 17 or other forms of hosiery (see FIG. 2B), and/or other wearable articles. It will be understood that the selected form may result in a pair of separately operating foot temperature measuring devices 1, such as a pad 15 integrated into an insole 16 article (FIG. 2A) or a pad 15 integrated into a sock 17 or other hosiery article (FIG. 2B). Where the temperature monitoring device 1 is a foot pad 15, the device 1 may be configured to be worn on a particular foot (i.e., on one of the left foot or the right foot, with an appropriate foot placement guide 12, 13) or universally (i.e., on either the left or right foot). The only limitation on form is that which will allow for the foot temperature measuring device 1 to take an accurate reading of the subject's body temperature at his or her foot/feet.

In various applications, the temperature sensors 4-11 need to be positioned and/or held within sufficient proximity to the subject's foot for a predetermined length of time in order to allow the temperature measuring device 1 to take an accurate reading. Thus, while a subject can satisfy the proximity and temporal duration requirements of the foot temperature measuring device 1 by standing on or placing their foot/feet on top of the foot temperature measuring device 1 while in a seated position, some subjects in need thereof may not be able to stand or sit up on their own or for very long comfortably or may prefer a wearable temperature measuring device 1. For these subjects, the foot pad 15 of the temperature measuring device 1 being integrated into a wearable article may allow the temperature measuring device 1 to be held against the foot/feet of the subject for at least the required temporal duration. The proximity and temporal duration requirements may be accomplished by manually holding or by employing straps (not shown) to hold the foot pad 15 of the temperature measuring device(s) 1 to the bottom of the subject's foot/feet or by using a wearable article (e.g., an insole 16 or a sock 17) to hold the foot pad 15 to the bottom of the subject's foot/feet. Integrating the temperature measuring device 1 into or coupling the temperature measuring device 1 with a wearable article (e.g., an insole 16 or a socket 17) may also allow for monitoring temperature of the subject's foot/feet during times of sleeping. In all cases, the temperature measuring device 1 is comfortable to the user/subject in need thereof while using the temperature measuring device 1.

As illustrated in FIGS. 1-2B, the plurality of temperature sensors 4-11 may be disposed on or near an instrument surface 18 of the temperature measuring device 1 such that the subject's temperature can be measured. The temperature sensors 4-11 may be directly coupled to the instrument surface 18, may be integrally formed with the instrument surface 18, or may be positioned below the instrument surface 18 (e.g., the temperature sensors 4-11 may be embedded within the foot temperature measuring device 1). The position of the temperature sensors 4-11 relative to the instrument surface 18 is configured such that the plurality of temperature sensors 4-11 are able to register accurate temperature readings of the subject's foot/feet.

The temperature monitoring device 1 may further include a protective coating 20. The protective coating 20 may be positioned over the instrument surface 18 of the foot temperature measuring device 1 and/or may be positioned over each of the individual temperature sensors 4-11. The protective coating 20 may be configured to protect each of the temperature sensors 4-11 from moisture (e.g., moisture on the foot/feet from after a bath/shower or from perspiration). It has been found that the plurality of temperature sensors 4-11 are more accurate when kept free of moisture such that the protective coating 20 may increase accuracy of readings. To prevent moisture from wet feet, the plurality of temperature sensors 4-11 may be protected from moisture by a protective coating 20.

Each of the plurality of temperature sensors 4-11 may be substantially flat. Providing the temperature sensors 4-11 as flat sensors may provide the user/subject with a comfortable experience while standing on or wearing the foot temperature measuring device 1. For example, each of the temperature sensors 4-11 may be configured as a thermistor-type temperature sensor 19. Thermistor-type temperature sensors 19 may be configured to provide accuracy in a variety of environmental conditions. In other examples, each of the temperature sensors 4-11 may be a thermocouple, a resistance thermometer, or known other electrical temperature sensor such as infrared body thermometers, temperature strips, digital heat sensors, infrared wave sensors, and basal thermometers. It will be understood that any type of temperature sensor may be used without departing from the scope of the present disclosure.

The plurality of temperature sensors 4-11 may be positioned in any pattern or order configured to achieve a substantially accurate temperature gauge of the subject's foot/feet. For example, as shown in FIG. 1, the plurality of temperature sensors 4-11 may be placed at contact points of a user's foot when the foot is positioned within one of the guides 12, 13, such as, but not limited to, the heel, ball, and toes (phalanges). For example, as shown in FIG. 1, the foot temperature measuring device 1 includes a temperature sensor 4 at midfoot-ball of left foot closest to the fifth metatarsal/phalange area, a temperature sensor 5 at the midfoot-ball of left foot first metatarsal/phalange area, a temperature sensor 6 at the front-left foot toe/phalange area, a temperature sensor 7 at the rear-left foot heel area, a temperature sensor 8 at the midfoot-ball of right foot first metatarsal/phalange area, a temperature sensor 9 at the front-right foot toe/phalange area, a temperature sensor 10 at the midfoot-ball of right foot fifth metatarsal/phalange area, and a temperature sensor 11 at the rear-right foot heel area. Any combination of one or more of these temperature sensors 4-11 may be used. It will be understood that any number of temperature sensors may be used without departing from the scope of the present disclosure.

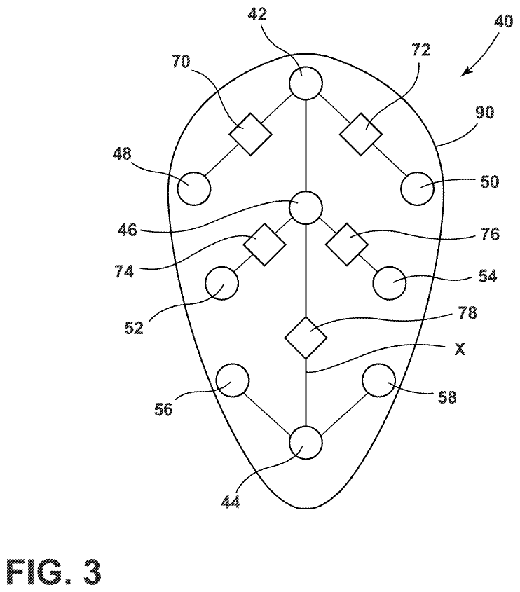

Referring now to FIG. 3, an alternative exemplary arrangement of a plurality of temperature sensors and a plurality of capacitive sensors is illustrated as a sensor assembly 40 for the temperature monitoring device 1. As shown in FIG. 3, the sensor assembly 40 for the may include a plurality of temperature sensors 42-58, a plurality of capacitive sensors 70-78, and a tactile feedback component 90. The plurality of temperature sensors and the plurality of capacitive sensors 70-78 may be positioned symmetrically about a centerline X. It will be understood that the plurality of temperature sensors 42-58 and the plurality of capacitive sensors 70-78 may be positioned asymmetrically about the centerline X or in any other pattern without departing from the scope of the present disclosure. The tactile feedback component 90 may be positioned about a periphery of the sensor assembly 40.

As illustrated in FIG. 3, the sensor assembly 40 may include a first temperature sensor 42 positioned proximate a first end of the centerline X, a second temperature sensor 44 positioned proximate a second end of the centerline X, and a third temperature sensor 46 positioned proximate the centerline X and between the first and second temperature sensors 42, 44. The sensor assembly 40 may further include fourth and fifth temperature sensors 48, 50 positioned on opposing sides of the first temperature sensor 42. In various examples, the fourth and fifth temperature sensors 48, 50 may be positioned offset from the first temperature sensor 42. The fourth and fifth temperature sensors 48, 50 may be aligned to be symmetrical about the centerline X or may be offset from each other. The sensor assembly 40 may further include six and seventh temperature sensors 52, 54 positioned on opposing sides of the second temperature sensor 44. In various examples, the six and seventh temperature sensors 52, 54 may be positioned offset from the second temperature sensor 44. The six and seventh temperature sensors 52, 54 may be aligned to be symmetrical about the centerline X may be offset from each other. The sensor assembly 40 may further include eighth and ninth temperature sensors 56, 58 positioned on opposing sides of the third temperature sensor 46. In various examples, the eighth and ninth temperature sensors 56, 58 may be positioned offset from the third temperature sensor 46. The eighth and ninth temperature sensors 56, 58 may be aligned to be symmetrical about the centerline X may be offset from each other.

In application, the plurality of temperature sensors 42-58 in various locations is configured to allow temperatures to be taken dynamically which may increase the accuracy of the temperature reading. To perform this dynamic reading, the temperature monitoring device 1 will first check and verify if the user's foot is covering a minimum of four sensors, as discussed in more detail elsewhere herein. Upon verification, all of the covered sensors will independently record the temperature and work together to collectively output the foot's temperature average. This type of dynamic reading and various locations of the sensors are configured to provide the opportunity to capture a reading from a varying range of foot sizes versus the standard one-foot size.

As introduced above, the sensors assembly 40 further includes a plurality of capacitive sensors 70-78. As shown in FIG. 3, the sensor assembly 40 may include first and second capacitive sensors 70, 72. The first capacitive sensor 70 may be positioned between the first temperature sensor 42 and the fourth temperature sensor 48, and the second capacitive sensor 72 may be positioned between the first temperature sensor 42 and the fifth temperature sensor 50. A third capacitive sensor 74 may be positioned between the third temperature sensor 46 and the sixth temperature sensor 52, and a fourth capacitive sensor 76 may be positioned between the third temperature sensor 46 and the seventh temperature sensor 54. A fifth capacitive sensor 78 may be positioned along the centerline X between the second and third temperature sensors 44, 46. However, it will be understood that the arrangement of the plurality of capacitive sensors 70-78 described above is exemplary only and may be altered or adjusted such that the capacitive sensors 70-78 may be in any other orientation within a guide 12, 13 (see FIG. 1) without departing from the scope of the present disclosure.

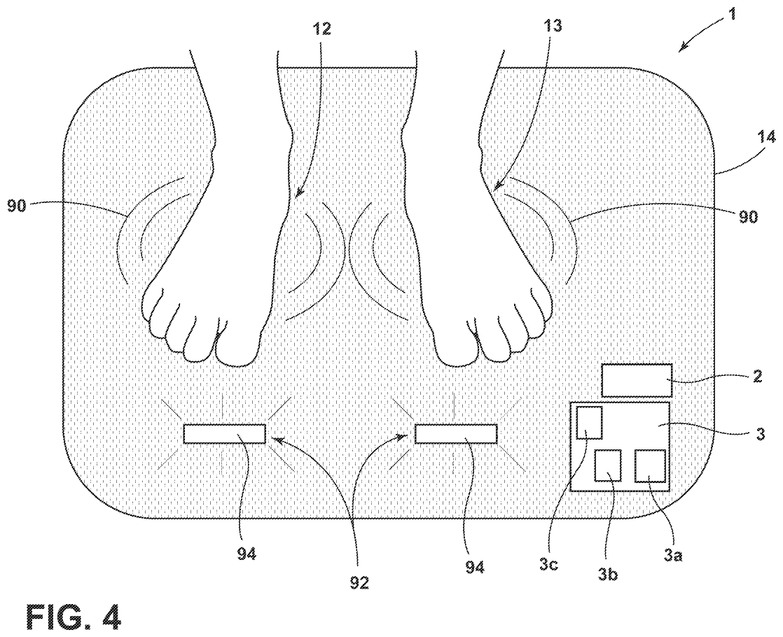

Referring now to FIGS. 3 and 4, the sensor assembly 40 may further include a tactile feedback component 90. The tactile feedback component 90 may be configured as a vibratory component positioned about a periphery of the sensor assembly 40. In other words, the tactile feedback component 90 may be positioned to outline a foot pad 15 or a guide 12, 13 of a platform 14 of the temperature monitoring device 1. The tactile feedback component 90 is configured to vibrate or otherwise convey direct vibrational pulses to provide feedback to a user. For example, the tactile feedback component 90 may be actuated when there is an irregularity in temperature readings or if a user's foot in not properly aligned within the guide 12, 13 or is not in contact with the temperature sensors 42-58. It will be understood that the tactile feedback component 90 may be utilized with any sensor distribution, including those shown in FIGS. 1-2B.

Referring now to FIG. 4, an exemplary temperature monitoring device 1 is illustrated including a platform 14. The platform 14 includes the tactile feedback component 90 and a visual feedback component 92. The visual feedback component 92 may include one or more light sources 94 positioned on or within the platform 14. For example, the light source 94 may be one or more LED strips positioned proximate a user's foot when the user's foot is positioned on the platform 14. When the light source 94 is positioned above or proximate the foot guide(s) 12, 13 or foot pad(s) 15 of the temperature monitoring device 1, a user is able to look down to the light source. In various examples, specific the colors of light may be configured to be associated with the different messages being displayed (e.g., a green light indicating that data was successfully captured, a flashing yellow light indicating a user should call a doctor, or a solid red light indicating no reading activity). As shown in FIG. 4, the visual feedback component 92 may be used in conjunction with the tactile feedback component 90. However, it will be understood that either feedback component 90, 92 may be used together or separately or with any other feedback option disclosed herein.

Referring now to FIGS. 1-4, in application, a user uses the temperature measuring device 1 by placing his or her foot/feet onto the instrument surface 18. In order to obtain an accurate temperature reading, the temperature measuring device 1 must be positioned and/or held within sufficient proximity to the subject's foot/feet for a predetermined length of time. A sufficient proximity should be understood to mean touching or near touching (directly or through a thin layer of clothing, such as socks or hosiery) with or without pressure onto the plurality of temperature sensors 4-11, 42-58. A predetermined length of time is any time period that is required or sufficient for the plurality of temperature sensors 4-11, 42-58 to measure and record an accurate temperature. In some embodiments, the predetermined length of time may be about two minutes or less, such as about 60 seconds to about 120 seconds, about 90 seconds to about 120 seconds, or about 60 seconds to about 90 seconds. In other embodiments, the predetermined length of time about one minute or less, such as about 30 seconds to about 60 seconds, about 45 seconds to about 60 seconds, or about 30 seconds to about 45 seconds. In still other embodiments, the predetermined length of time may be about 30 seconds or less, such as about 1 second to about 30 seconds, about 15 seconds to about 30 seconds, about 1 second to about 15 seconds, about 1 second to about 5 seconds, about 5 seconds to about 10 seconds, or about 10 seconds to about 15 seconds. For example, the predetermined length of time may be about 15 seconds.

Referring now to FIGS. 1 and 4, the plurality of temperature sensors 4-11 or 42-58 may be in electric communication with a power supply 2 and a controller 3. The power supply 2 can be any source of voltage to power the plurality of temperature sensors 4-11, 42-58 and the controller 3, which is discussed in more detail elsewhere herein. For example, the power supply 2 may be a battery, such as, for example, a 9V battery 2. In various examples, the battery 2 may be rechargeable. In other examples, the power supply 2 is a 120V alternating current, such as available in electric outlets. The temperature measuring device 1 may also include an on/off switch (not shown) that is accessible to a user/subject without the need for tools.

The temperature measuring device 1 may be configured to save power in order to conserve battery life, when so provided. For example, the temperature measuring device 1 can be kept in an “off” or low power “sleep” mode under normal conditions. In various examples, the temperature measuring device 1 may “wake up” periodically to an “on” condition at predetermined (programmed time periods stored within a memory 3a) in order to take a reading before returning to the “off” or “sleep” mode. In other examples, the temperature measuring device 1 may be configured to trigger an “on” condition when the user/subject provides a triggering amount of pressure (trigger pressure can be programmed and stored within the memory 3a) so that a reading measurement is only taken when the user/subject is well within the proximity sufficient for an accurate reading. Where the temperature measuring device 1 is configured to trigger an “on” condition when provided with a triggering amount of pressure, the device 1 may further include a predetermined time duration between data collection (time period can be programmed and stored within the memory 3a). For example, if the user/subject is standing on a platform 14 of the device 1 while performing some task, the device 1 can be triggered to only make a reading every two minutes, five minutes, 10 minutes, 15 minutes, etc.

Each of the plurality of temperature sensors 4-11, 48-52 may be in electric communication with the controller 3. An output of an electronic signal carrying the temperature reading information is transferred from the plurality of temperature sensors 4-11, 48-52 to the controller 3. The controller 3 processes (via a processor 3b, microcontroller, CPU, or similar device well-known in the art) the electronic signal from the plurality of temperature sensors 4-11, 48-52 to extrapolate the temperature reading from each of the plurality of temperature sensors 4-11, 48-52. The temperature readings form the plurality of temperature sensors 4-11, 48-52 are stored in a memory 3a that is in electronic communication with the processor 3b. In various examples, the temperature readings data are given a time and date stamp according to their time of creation, and this information is stored in association with the temperature readings data.

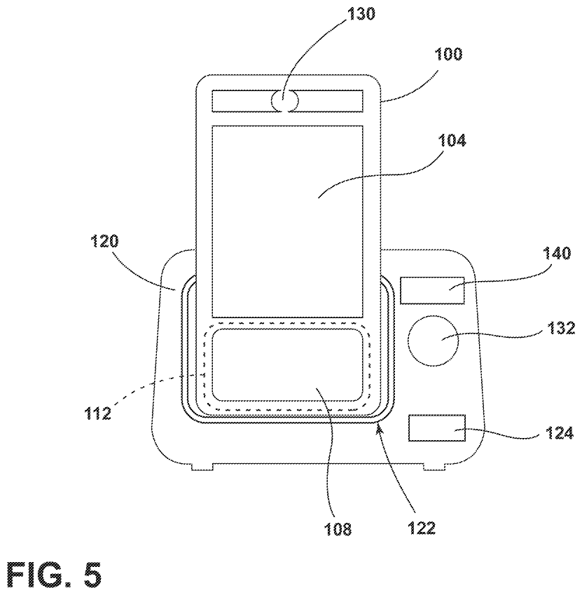

The memory 3a may be a removable memory storage device, such as a flash memory device (an SD card, a USB memory storage device, or the like). The memory 3a can be accessed by the processor 3b in order to transfer the data stored therein to another computing device, as discussed in more detail elsewhere herein. The method of transfer can be any known method within the field of medical devices. For example, the data can be recalled and transferred to a hardware device, such as a flash memory device (an SD card, a USB memory storage device, or the like) or a portable companion device 100 (see FIG. 5). For example, the processor 3b may be in electronic communication with an electromagnetic transceiver 3c for wirelessly transferring the stored data. In some examples having an electromagnetic transceiver 3c, the memory 3a can be omitted for direct transmission and storage of the data on a separate computing device's memory.

The foot temperature measuring device 1 may be integrated directly with a doctor's office or medical establishment (e.g., a hospital or nursing home) by an Internet connection or a direct network connection to transfer the captured data directly to the computing device(s) thereof for convenient analysis by caregivers. In various examples, the data may be transferred to a handheld computing device (e.g., a smartphone or tablet) or to a portable companion device 100 with an app for collecting, analyzing, and/or storing the data.

Referring now to FIG. 5, a portable companion device 100 is illustrated. The portable companion device 100 is configured to be in communication with the temperature monitoring device 1. For example, the portable companion device 100 may be in communication with the temperature monitoring device 1 using Bluetooth technology. The portable companion device 100 may be configured to store, map, and display a user's trending data from the temperature monitoring device 1, securely send the trending data to a physician team or group, and/or provide conjunctive feedback communication methods for a user (e.g., visual, auditory, or tactile feedback). In other words, the portable companion device 100 may be configured to store trending data with a timestamp as it is received from the controller 3 of the temperature monitoring device 1, check the trending data for any temperature variances, and/or provide messages for the temperature monitoring device 1 to output to a user via visual and/or tactile feedback components. The portable companion device 100 may further be configured to securely sync and send trending data to physician secure network via Wi-Fi or 4G/5G data technology according to HIPPA regulation and/or receive secure instructional/additional communication messages from physicians/nurse for foot readings (e.g., a message to increase foot reading 3 times a day or a message to take 2 foot readings 5 minutes within each other for the next 7 days). The portable companion device 100 may further be configured to schedule foot reading reminders and receive notification alerts. For example, the portable companion device 100 may be configured to provide a scheduled reminder notification call or text to user's phones to remind them to capture their foot temperature via an automated phone call or text messaging system.

As illustrated in FIG. 5, the portable companion device 100 may include a display 104 configured to allow a user to view data and configure the device 100. The display 104 may further be configured to display messages for a user regarding the data. In various examples, the display 104 may be configured as a touch screen. In other examples, the device 100 may include one or more button controls 108 for operating the device 100. The portable companion device 100 may be operable using a power source 112 (e.g., a battery).

A base 120 may be configured to at least partially receive the portable companion device 100. For example, the base 120 may define a receiving space 122 configured to receive the portable companion device 100. In various examples, when the portable companion device 100 is received by the base 120, the portable companion device 100 may be electrically coupled with the base 120 to charge the power source 112. The base 120 may further be configured to be in wireless communication with the portable companion device 100. For example, a locator button 124 may be positioned on the base 120. When the locator button 124 is actuated, the base 120 may be configured to communicate with the portable companion device 100 to indicate the location of the device 100 (e.g., via auditory or visual feedback).

The portable companion device 100 may include a speaker 130, or other auditory feedback component. The speaker 130 may be configured to provide feedback to a user regarding the temperature reading or to provide a location of the device 100 to a user. A second speaker 132 may be positioned on the base 120. However, it is contemplated that the base 120 may be used without the second speaker 132 without departing from the scope of the present disclosure.

The portable companion device 100 may further include a light source 136 configured to provide visual feedback to a user. For example, the light source 136 may be an LED configured to provide visible flashing messages to a user. The portable companion device 100 may further include a tactile feedback component (not shown) configured to provide vibratory alerts to a user. Each of the light source 136 and the tactile feedback component may be configured to sync with the tactile feedback component 90 and the visual feedback component 92 of the temperature monitoring device 1 or may be operated independently from the visual and tactile feedback components 90, 92. In various examples, the base 120 may further include a second light source 140 configured to operate in conjunction with the light source 136 of the device and/or the visual feedback component 92 of the temperature monitoring device 1. However, it is contemplated that the base 120 may be used without the second light source 140 without departing from the scope of the present disclosure.

Data stored within the portable companion device 100 may be protected via a user credential requirement (e.g., a passcode or PIN entered into the portable companion device 100. In other examples, the data may be protected using a biometric passcode configured to prompt verification of a user's footprint via the plurality of capacitive sensors 70-78. Because capacitive sensors work by using arrays of tiny capacitor circuits to collect data, by creating a large enough array of capacitors (e.g., the plurality of capacitive sensors 70-78), an accurate and highly detailed image of the ridges of a footprint (much like a fingerprint) can be created from the electrical signals. Additionally, the capacitive sensors 70-78 may be configured to aid the temperature sensors 42-58 in checking for a proper circuit (or blood flow) within the limb. In the event that there is little to no circuit being detected by the capacitive sensors 70-78, the portable companion device 100 may be configured to provide an alert to the user urging the user to contact their physician immediately as the mat does not detect a circuit.

It has been found that a subject's lower extremity temperature correlates well with blood flow where a decrease in blood flow relates to a decrease in temperature of a subject's foot. For example, a drop of four degrees Fahrenheit from a baseline temperature in one or both feet of a subject may indicate a period of decreased blood flow that could cause tissue damage complications for the subject's lower extremities if not acted upon. A baseline temperature may be an average baseline temperature preprogrammed into memory 3a, or it may be a personalized baseline temperature that may be programmed into memory 3a for the user/subject using the device 1. Therefore, the temperature data captured can be used by a caregiver of the subject, such as a nurse or doctor, to monitor the health of the subject and/or prescribe preventive/remedial action or corrective action based on the analysis of the transferred data. The data captured can be processed and stored for each individual sensor of the plurality of temperature sensors 4-11, 48-52. The captured data can also be processed to provide an averaged temp for all sensors for one or both feet of the subject. The data captured can be processed to glean trends in temperature and blood flow for each foot and/or for each sensor.

By monitoring the temperature of the lower extremities of a subject, a foot or a portion thereof that is experiencing periods of lower temperature and lower blood flow can be found before tissue damage and ulceration occurs. The foot temperature measuring device 1 may act as an early warning device and, thereby, may provide for a prevention of complications of diabetes, low blood flow, and/or low temperature of a lower extremity. In addition to using the data captured to diagnose and take remedial action(s), the data can alert the user/subject in need thereof by performing an audible or visual alarm when the temperature reading of any foot or individual temperature sensor falls below the baseline temperature.

The foot temperature measuring device 1 is convenient to use and care for. The foot temperature measuring device 1 is light weight, mobile, and easily transportable. The foot temperature measuring device 1 is waterproof for cleaning. The platform 14 can be cleaned by wiping away soiling. Wearable forms are also capable of cleaning with water. For example, the foot pad 15 of the insole 16 may be removed from shoes or other footwear for cleaning and/or the foot pad 15 of the sock/hosiery-type articles 17 may be hand-washable with waterproof sealing of all electronic components and/or removable electronic components.

In some examples, the device 1 may be configured to determine a body mass index of a user/subject with mass/weight scales built into the platform 14 of the device 1 when coupled to a data for the user's/subject's height data (which may be preprogrammed into the memory 3a). The device 1 may also have a percent body fat measurement capability. In various examples, the device 1 may be configured to integrate with other health apps, platforms, and systems. Alternative embodiments include providing a plurality of temperature sensors 4-11, 48-52 for placement on a flat surface or directly on the user's/subject's skin surface or sock/hosiery article of the foot bottom.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

What is claimed is:

1. An electronic temperature measuring device comprising:
a base;
a temperature sensor positioned on the base;
a capacitive sensor positioned on the base; and
a controller configured to receive input from at least one of the temperature sensor and the capacitive sensor.

2. The electronic temperature measuring device of claim 1, wherein the base includes one or more light sources, and wherein the controller is configured to selectively actuate the one or more light sources in response to input from at least one of the temperature sensor and the capacitive sensor.

3. The electronic temperature measuring device of claim 1, wherein at least one of the temperature sensor and the capacitive sensor includes a protective coating.

4. The electronic temperature measuring device of claim 1, wherein the base is integrally formed with a wearable article.

5. The electronic temperature measuring device of claim 1, further comprising a tactile feedback component configured to be selectively actuated by the controller in response to input from at least one of the temperature sensor and the capacitive sensor.

6. An electronic temperature measuring device, comprising:
a base;
a plurality of temperature sensors positioned on the base, wherein the plurality of temperature sensors symmetrically disposed about centerline of the base; and
a controller configured to receive input from the plurality of temperature sensors.

7. The electronic temperature measuring device of claim 6, further comprising:
a plurality of capacitive sensors operably coupled with the controller, wherein the controller is configured to receive input from the plurality of capacitive sensors.

8. The electronic temperature measuring device of claim 7, further comprising:
a tactile feedback component configured to be selectively actuated by the controller in response to input from at least one of the plurality of temperature sensors and the plurality of capacitive sensors.

9. The electronic temperature measuring device of claim 6, further comprising:
a feedback device communicatively coupled with the controller, wherein the feedback device includes a processor having a memory, and wherein the feedback device is configured to receive data from the controller to be stored on the memory of the feedback device.

10. The electronic temperature measuring device of claim 9, wherein the feedback device is configured to selectively transmit the data from the controller to a remote user.

11. The electronic temperature measuring device of claim 9, wherein the feedback device includes a speaker configured to selectively provide auditory feedback in response to input from the controller.

12. The electronic temperature measuring device of claim 9, wherein the feedback device includes a light source configured to selectively provide visual feedback in response to input from the controller.

13. The electronic temperature measuring device of claim 9, wherein the feedback device includes a tactile feedback component configured to selectively provide tactile feedback in response to input from the controller.

14. The electronic temperature measuring device of claim 6, wherein the base is a foot pad configured to be insertable into a wearable article.

15. The electronic temperature measuring device of claim 6, wherein the base is a platform.

16. The electronic temperature measuring device of claim 15, wherein the platform includes, and is configured to provide feedback to a user through, one of a tactile feedback component, a light source, and a speaker.

17. A method of assembling a temperature monitoring device, comprising:
positioning a plurality of temperature sensors on a substrate such that the plurality of temperature sensors are symmetrically disposed about a centerline of the substrate;
positioning a plurality of capacitive sensors on the substrate such that the plurality of capacitive sensors are symmetrically disposed about the centerline of the substrate;
operably coupling the plurality of temperature sensors and the plurality of capacitive sensors with a controller such that the plurality of temperature sensors and the plurality of capacitive sensors are configured to provide input the controller.

18. The method of claim 17, further comprising:
integrally forming the substrate with a wearable article.

19. The method of claim 17, further comprising:
integrally forming the substrate with a platform.

20. The method of claim 17, further comprising:
communicatively coupling the controller with a portable feedback device.
